(19) United States
(12) Patent Application Publication
(10) Pub. No.: US 2011/0295110 A1
(43) Pub. Date: Dec. 1, 2011

(54) METHOD AND SYSTEM OF TRACKING AND MAPPING IN A MEDICAL PROCEDURE

(75) Inventors: Robert Manzke, Sleepy Hollow, NY (US); Raymond Chan, San Diego, CA (US)

(73) Assignee: KONINKLIJKE PHILIPS ELECTRONICS N.V., EINDHOVEN (NL)

(21) Appl. No.: 13/148,050

(22) PCT Filed: Jan. 18, 2010
(86) PCT No.: PCT/IB2010/050226

§ 371 Date: Aug. 5, 2011

Related U.S. Application Data
(62) Division of application No. 61/151,593, filed on Feb. 11, 2009.

(51) Int. Cl. A61B 5/05

(52) U.S. Cl. 600/424

(57) ABSTRACT

A tracking system for a target anatomy of a patient can include a medical device having a magnetic source (299) integrally connected thereto and positionable in the target anatomy, a plurality of Optical Atomic Magnetometer (OAM) sensors (210) for detecting a magnetic field from the magnetic source, and a processor (120) for determining a position of the medical device based on one or more parameters associated with the detected magnetic field. Other embodiments can include connecting a magnetic source with the medical device and having the OAM sensors positionable in proximity to the target anatomy.

The present application relates to the therapeutic arts, in particular to tracking and mapping for medical procedures and will be described with particular reference thereto.

Various techniques and systems have been proposed to improve the accuracy of instrumentality placement (e.g., catheter placement) into tissue based on measurements from 3D imaging formats. These imaging formats attempt to locate the needle entry device in relation to therapy-targeted tissue, such as MRI detected target tissue. These imaging formats generate imaging data that are used to determine the appropriate positioning of the needle during treatment, where the needle is typically placed in a guide device and moved into the tissue.

In many cases, the medical device is delivered solely on the basis of this imaging data information, and confirmation of the final medical device position relative to the target requires a second set of images to be acquired. In cases where tissue stiffness variations are extreme, the medical device may deviate from the desired path. Similarly, the medical device may distort the tissue itself and thereby move the target tissue to a new location, such that the original targeting coordinates become inaccurate.

Accordingly, there is a need for a technique and system for accurately placing surgical devices in a target anatomy during a medical procedure. There is a further need for a technique and system for accurately mapping target anatomy

This Summary is provided to comply with U.S. Rule 37 C.F.R. §1.73, requiring a summary of the invention briefly indicating the nature and substance of the invention. It is submitted with the understanding that it will not be used to interpret or limit the scope or meaning of the claims.

In accordance with one aspect of the exemplary embodiments, a method of tracking in a medical procedure can include providing a medical device with at least one Optical Atomic Magnetometer (OAM) sensor integrally connected thereto; positioning the medical device into a target anatomy of a patient; applying a magnetic field to the target anatomy; detecting the magnetic field using the OAM sensor; and determining a position of the medical device based on one or more parameters associated with the detected magnetic field.

In accordance with another aspect of the exemplary embodiments, a computer-readable storage medium can include computer-executable code stored therein, where the computer-executable code is configured to cause a computing device, in which the computer-readable storage medium is provided, to determine a position of a medical device in a target anatomy of a patient based on one or more parameters associated with a magnetic field applied to the target anatomy and detected using Optical Atomic Magnetometer (OAM) sensors.

In accordance with another aspect of the exemplary embodiments, a tracking system for a target anatomy of a patient can include a medical device having a magnetic source integrally connected thereto and positionable in the target anatomy; a plurality of Optical Atomic Magnetometer (OAM) sensors for detecting a magnetic field from the magnetic source; and a processor for determining a position of the medical device based on one or more parameters associated with the detected magnetic field.

In accordance with another aspect of the exemplary embodiments, a method of mapping is provided including introducing magnetic particles into a patient; providing a medical device with at least one Optical Atomic Magnetometer (OAM) sensor integrally connected thereto; positioning the medical device in proximity to a target anatomy (105) of the patient; detecting the magnetic field of the magnetic particles using the OAM sensor; and mapping the target anatomy based on one or more parameters associated with the detected magnetic field.

The exemplary embodiments described herein have a number of advantages over contemporary systems and processes, including accuracy of surgical device placement. Additionally, the system and method described herein can be utilized through retrofitting existing surgical devices. Still further advantages and benefits will become apparent to those of ordinary skill in the art upon reading and understanding the following detailed description.

The above-described and other features and advantages of the present disclosure will be appreciated and understood by those skilled in the art from the following detailed description, drawings, and appended claims.

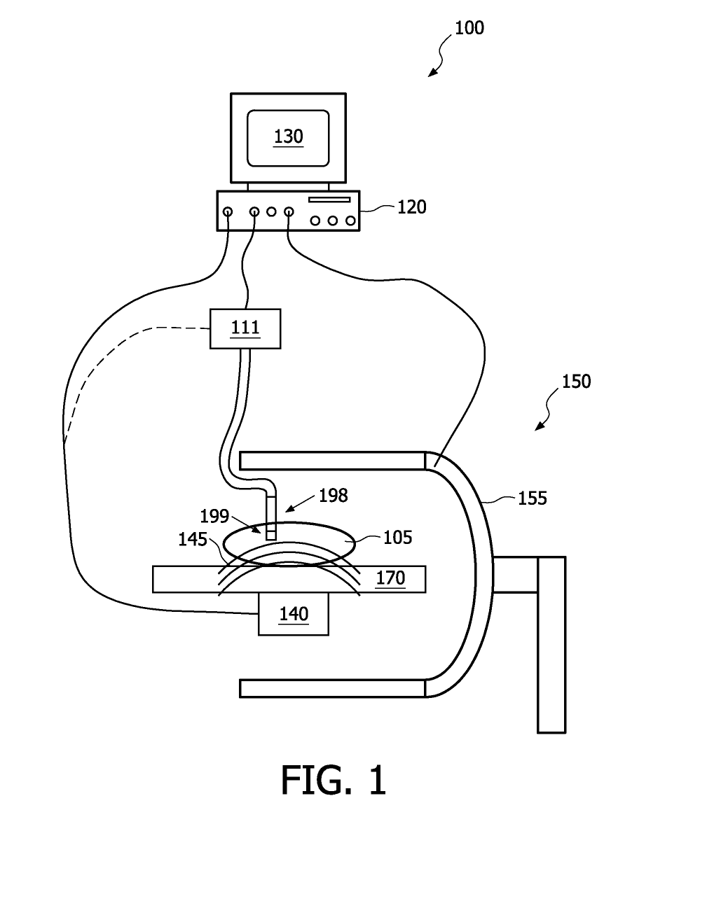

FIG. 1 is a schematic illustration of a tracking system according to one exemplary embodiment for use in a medical procedure;

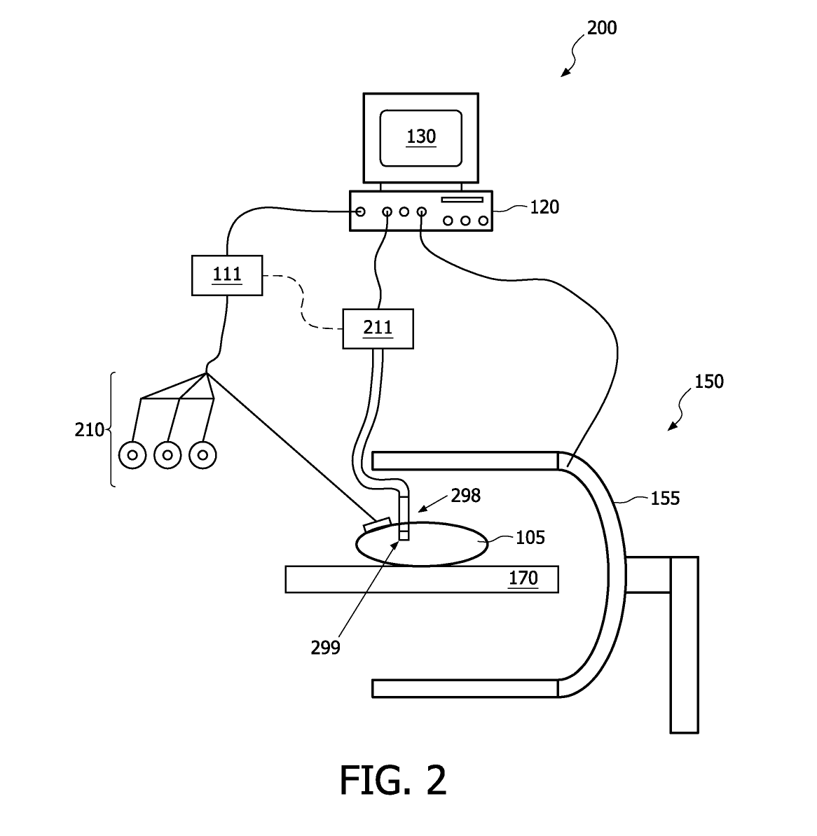

FIG. 2 is a schematic illustration of another tracking system according to another exemplary embodiment;

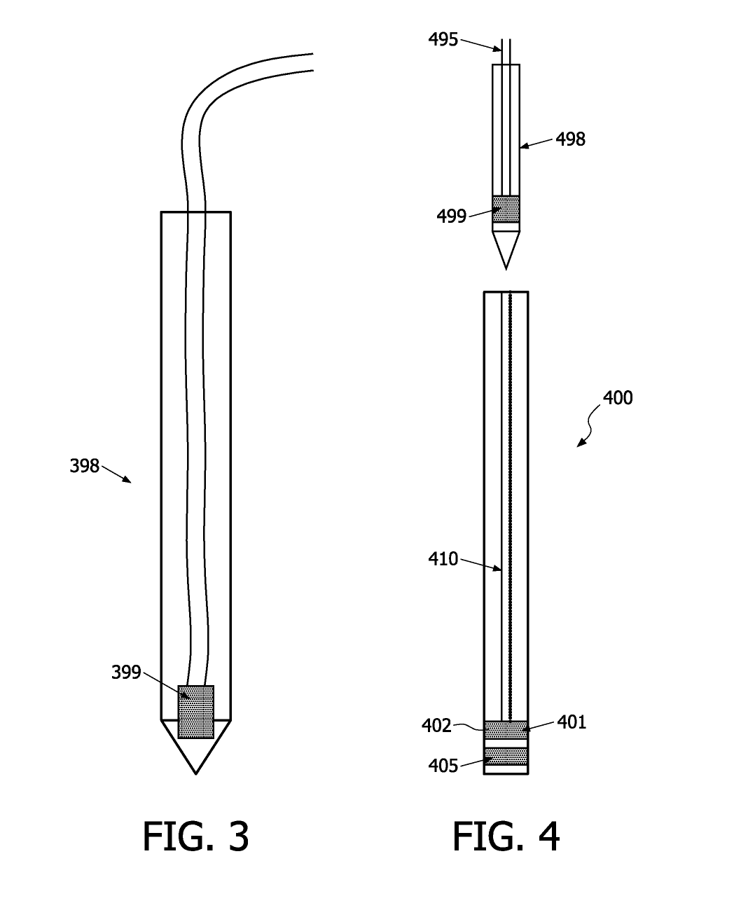

FIG. 3 is a schematic illustration of a surgical device for use with the tracking systems of FIGS. 1 and 2;

FIG. 4 is a schematic illustration of another surgical device for use with the tracking systems of FIGS. 1 and 2; and

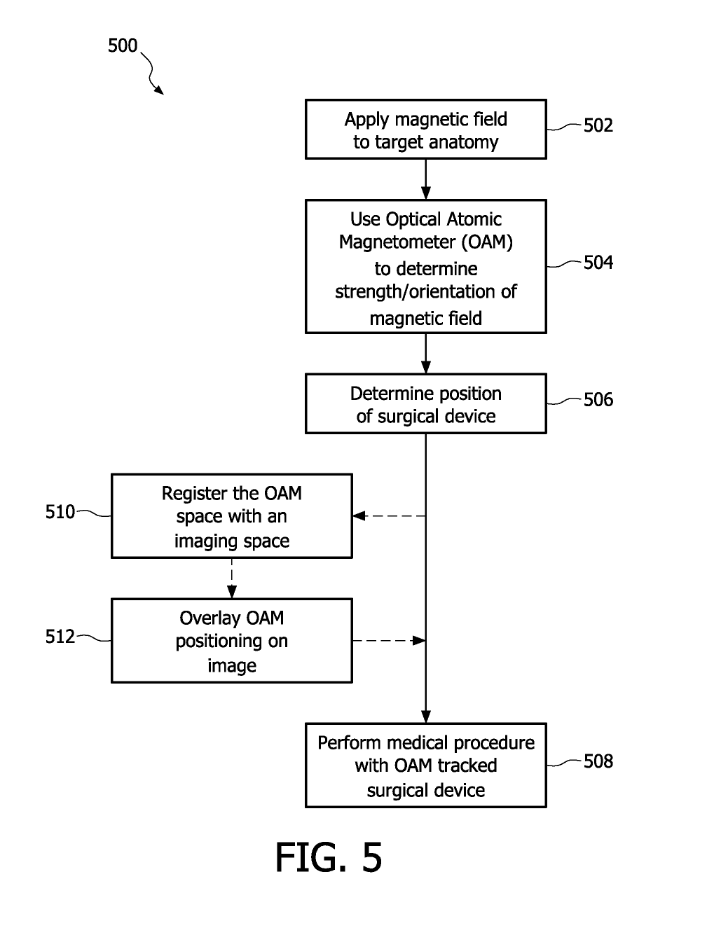

FIG. 5 is a method that can be used by the system and devices of FIGS. 1-4 for performing tracking and/or mapping during a medical procedure.

The exemplary embodiments of the present disclosure are described with respect to an Optical Atomic Magnetometer (OAM) tracking system for a surgical or other medical device to be utilized during a procedure for a human. It should be understood by one of ordinary skill in the art that the exemplary embodiments of the present disclosure can be applied to, and utilized with, various types of medical or surgical devices, various types of procedures, and various portions of the body, whether human or animal. The exemplary embodiments can also be used for mapping electro-magnetic activations in or around the target anatomy using a surgical device such as a catheter or needle inserted into or in proximity to the target anatomy. The exemplary embodiments are described herein as using OAM tracking in combination with imaging, but the present disclosure contemplates the OAM tracking of a surgical device being used without an imaging modality. The use of the method and system of the exemplary embodiments of the present disclosure can be adapted for application to other types of tracking and/or mapping in a target anatomy.

Referring to FIG. 1, a tracking system 100 is shown which can have a surgical device 198, such as a catheter or needle, with an Optical Atomic Magnetometer (OAM) sensor 199 connected thereto. The OAM sensor 199 can be positioned along various portions of the surgical device 198, such as the tip of the surgical device. While the exemplary embodiment shows a single OAM sensor 199, the present disclosure contemplates the use of any number of OAM sensors that can be in various configurations along the surgical device 198. The surgical device 198 can be utilized in the target anatomy 105.

The OAM sensor 199 can be a highly sensitive magnetometer capable of detecting magnetic fields 145, such as generated by a magnetic field generator 140. Sensor 199 can utilize a measurement of the Larmor precession of spin-polarized atoms in the magnetic field in order to achieve magnetic field detection, including of extremely low strength magnetic fields. For example, alkali atoms have an unpaired electron in their outer shell whose spin rapidly precesses in an external magnetic field. A resonant or pump laser beam can be applied through an atomic vapor cell to orient the atomic spins, and a probe laser beam can be applied to and pass through the vapor to detect how the spins react to the magnetic field (such as detecting their polarization which varies depending on the magnetic field). In one embodiment, the OAM sensor 199 can utilize polarized alkali-metal vapor (such as Potassium, Rubidium and/or Cesium).

The OAM sensor 199 can be operably connected to an OAM controller 111. The OAM controller 111 can include a laser source and/or a measurement unit. For example, the controller 111 can provide first laser energy (the resonant or pump laser) and second laser energy (the probe laser) to the OAM sensor 198 through fiber optic lines or other connections. The controller 111 can be in communication with a processor 120, such as a computer workstation, and can communicate the magnetic field data, such as intensity and orientation, to the processor. In one embodiment, the controller 111 can process the intensity and orientation data, and can provide measurement information to the processor 120. In another embodiment, the controller 111 can transmit raw data to the processor 120, which then analyzes the raw data to determine a position of the OAM sensor 199 with respect to the target anatomy 105.

In one embodiment, the field generator 140 can produce a spatially-dependent static or dynamic magnetic field which is applied to the target anatomy 105. The field generator 140 can be positioned in proximity to the target anatomy 105, such as under a bed 170 or other support structure for the patient, although various locations of the field generator are contemplated by the present disclosure. Since the magnetic field in the target anatomy is spatially-encoded, a detection of the intensity and orientation of the field by the OAM sensor 199 allows for a spatial determination of the sensor and thus the surgical device 198.

In one embodiment, tracking system 100 can be used with, or can include, an imaging modality 150, such as a high resolution imaging modality, including an x-ray scanner 155. For example, a high resolution image of the target anatomy 105, including the surgical device 198 and the surrounding region (e.g., tissue, organs, vessels, and so forth) can be generated by the scanner 155 and stored in an image memory. The image memory can be incorporated into workstation 120 and/or can be a separate storage and/or processing device. A C-arm x-ray scanning device 155 is shown in FIG. 1 for illustrative purposes, but the present disclosure contemplates the use of various imaging devices, including a closed device, open MRI and so forth. The present disclosure contemplates the use of various imaging modalities, alone or in combination, including MRI, ultrasound, X-ray, CT, and so forth. The present disclosure also contemplates the imaging modality 150 being a separate system that is relied upon for gathering of images, including pre-operative and/or intra-operative images. The computer workstation 120 can utilize the OAM data from sensor 199 for registration of the magnetic space with the imaging space of imaging modality 150.

The resulting registration of the magnetic space with the imaging space can then be utilized intra-operatively for tracking of surgical device 198 that includes OAM sensor(s) 199. The registration can be utilized to transfer the OAM measurements of the surgical device sensor 199 from the magnetic frame of reference to the image frame of reference, which can be displayed by display device 130. In one embodiment, the display of the surgical device 198 through use of OAM tracking and imaging can be in real-time. In another embodiment, system 100 can register the OAM measurements of the sensor 199 to the frame of reference of the image without user intervention. In another embodiment, system 100 can graphically display the OAM measured positioning overlaid or super-imposed onto the image, such as through use of display 130. In one embodiment, the user can accept, reject, or edit the registered OAM measurements of the positions as an accurate registration, and then proceed with the surgical procedure.

The present disclosure contemplates other techniques being utilized in addition to the OAM tracking of surgical device 198. For example, the exemplary embodiments can utilize image correlation or processing algorithms for localization. For instance, one or more features that appear in the image and have a known position can be utilized by the image correlation algorithms, such as portions of the surgical device 198.

In one embodiment, tracking system 100 can also include one or more fiducial markers, which can be mounted or otherwise positioned in proximity to the target anatomy 105 such as external to the patient on his or her body. Each marker can include a sensor unit, such as an OAM sensor 199, which is in communication with the processor 120. In one embodiment, the fiducial markers can comprise a material that is visible during imaging. The OAM sensor 199 of surgical device 198 and the fiducial markers can provide position and orientation information to the processor 120 based on detection of the magnetic field 145, and can facilitate the registration and tracking process for the surgical device 198.

Referring to FIG. 2, another exemplary embodiment of a tracking system is shown and generally represented by reference numeral 200. Tracking system 200 can include one or more components described with respect to tracking system 100, including the processor 120. Tracking system 200 can include a surgical device 298 having a magnetic source or field generator 299 thereon. Similar to the surgical device 198 described above, surgical device 198 can be positioned in the target anatomy 105 during a medical procedure and tracked utilizing OAM techniques.

OAM sensors 210 can be placed on the patient in proximity to the target anatomy 105. The particular number and configuration of the OAM sensors 210 can vary. The OAM sensors 210 can be operably connected to the OAM controller 111 for receiving the first laser energy (the resonant or pump laser) and second laser energy (the probe laser) in order to detect the intensity and orientation of the magnetic field generated by magnetic source 299 of the surgical device 298. In one embodiment, the magnetic source 299 can be operably connected to a magnetic field driver 211, although the present disclosure contemplates generating the magnetic field using various techniques, including a permanent magnet positioned on the surgical device 298, current-fed coils positioned along the surgical device, and so forth. Similar to tracking system 100, the imaging modality 150 can be utilized to facilitate the tracking procedure, including registration of the magnetic space with the imaging space, although the present disclosure contemplates utilizing only the OAM space for tracking the surgical device 298.

Referring to FIG. 3, a surgical device 398 is shown that can be utilized with either of the tracking systems 100 and 200. The surgical device 398 includes a sensing component 399, which can be an OAM sensor if the surgical device is being used with tracking system 100 or can be a magnetic source if the surgical device is being used with tracking system 200. The particular positioning of the sensing component 399 can vary, including being in proximity to a tip of the surgical device 398. Transmission lines, such as fiber optics, can be connected to the sensing component 399 to place the sensing component in communication with an OAM controller if the surgical device is being used with tracking system 100 or to place the sensing component in communication with a magnetic field driver if the surgical device is being used with tracking system 200. The particular type of the surgical device 398 can vary, including catheters, needles and so forth, and is not intended to be limited.

Referring to FIG. 4, first and second surgical devices 400 and 498 are shown that can be utilized with either of the tracking systems 100 and 200. The surgical devices 400 and 498 include sensing components 401 and 499, which can be OAM sensors if the surgical devices are being used with tracking system 100 or can be magnetic sources if the surgical devices are being used with tracking system 200. The particular positioning of the sensing components 401 and 499 can vary, including in proximity to the tips of the surgical devices 400 and 498.

Transmission lines 410 and 495, such as fiber optics, can be connected to the sensing components 401 and 499 to place the sensing components in communication with an OAM controller if the surgical devices are being used with tracking system 100 or to place the sensing components in communication with a magnetic field driver if the surgical devices are being used with tracking system 200. In one embodiment, the first surgical device 400 can be a catheter that allows positioning of the second surgical device 498 (such as a needle) therethrough. Tracking systems 100 and/or 200 can allow a user to track the positioning of both surgical devices 400 and 498 during a surgical procedure, including with respect to each other.

In one embodiment, the surgical device, such as catheter 400, can have an imaging band 405 or other identification area. The band 405 can be made of a material, and have a size and shape, that allows it to be visible during imaging of the target anatomy. The particular type of material, as well as its size and shape, can be based on a number of factors, including the type of imaging that will be utilized, and the target anatomy that is being imaged. For instance, the band 405 can comprise a gadolinium-doped material where the imaging modality is magnetic resonance imaging. As another example, the band 405 can comprise a plastic or bone-like substance of sufficient density to provide for X-ray attenuation where the imaging modality is computer tomography or X-ray imaging. In one embodiment, the band 405 can be positioned in proximity to the OAM sensor or magnetic source. The band 405 can be visible in the imaging, which facilitates various registration techniques between the magnetic and imaging spaces, including point-by-point registration.

Referring to FIG. 5, a method 500 of OAM tracking in a medical procedure is shown. Method 500 can be employed for various types of medical treatments where positioning of a medical device is a desired criteria of the procedure. In step 502, a spatially-encoded magnetic field can be applied to the target anatomy, such as from an external field generator or an internal magnetic source. In step 504, one or more OAM sensors can be utilized to detect the generated magnetic field and determine the intensity and orientation of the generated magnetic field.

In step 506, the position of the surgical device can be determined based on the intensity/orientation data obtained by the OAM sensor(s). For example, the OAM sensor can be affixed to the surgical device and its position can be detected based on one or more parameters of the magnetic filed at that position. As another example, OAM sensors can be positioned external to the patient and can determine the position of the surgical device based on the magnetic field being generated by the magnetic source positioned on the surgical device. In step 508, the medical procedure can be performed using the OAM data, which is analyzed to determine a position and/or orientation of the surgical device and thus track the surgical device throughout the medical procedure.

In one embodiment in step 510, imaging data can be obtained with respect to the target anatomy and the OAM space can be registered with the imaging space. Various registration techniques can be utilized, including point-to-point registration. Fiducial markers, imageable portions of the surgical device and the like can be utilized to facilitate the registration process. In step 512, the position information for the surgical device derived from the OAM techniques can be overlayed on the images to provide a visual representation of the surgical device with respect to the target anatomy.

In one embodiment, one or more other OAM sensors can be positioned in a gradiometric configuration at a distance from the magnetic source to detect any interference magnetic fields. The detected interference fields can then be compared to the detected magnetic field in the target anatomy 105 to cancel the interference and provide for a more accurate determination of the intensity and orientation of the field. In another embodiment, the surgical device and OAM sensor connected thereto can utilize the localized magnetic field (Earth magnetic field) as the magnetic source without the need for a field generator. In one embodiment, metal field distortion correction or compensation can be applied to the OAM data.

In one embodiment, the surgical device and OAM sensor connected thereto can map the magnetic field being generated within the target anatomy. For example, neural or cardiac target anatomies can be entered using the surgical device with OAM sensor, and magnetic fields and gradients therein can be measured.

The OAM sensor sensitivity can vary and can be in the picotesla range or lower. In one embodiment, sensors detecting magnetic fields in the picotesla range can be utilized in combination with areas that have low noise, e.g., EM quiet measurement rooms.

In another embodiment, the OAM sensors can be utilized for tracking magnetic materials in the body. For example, magnetic nanoparticles can be introduced into the body which can attach to target regions, such as particular cells or body locations. The OAM sensors can then be used for determining the location of the magnetic nanoparticles and mapping a target region.

In one embodiment, a temperature-control mechanism can be provided with the OAM sensor to keep the optically sensitive vapor in a vapor state. For example, a temperature of about 100° C. can be utilized but can vary depending on the vapor utilized. At elevated temperatures, the target tissue can be ablated if the heated vapor capsule comes into contact with the tissue. In one embodiment, a temperature control device, such as an insulation layer 402 in FIG. 4, can be operably coupled to the OAM sensor to thermally isolate the vapor capsule from surrounding organs, protecting tissue from thermal damage.

The invention, including the steps of the methodologies described above, can be realized in hardware, software, or a combination of hardware and software. The invention can be realized in a centralized fashion in one computer system, or in a distributed fashion where different elements are spread across several interconnected computer systems. Any kind of computer system or other apparatus adapted for carrying out the methods described herein is suited. A typical combination of hardware and software can be a general purpose computer system with a computer program that, when being loaded and executed, controls the computer system such that it carries out the methods described herein.

The invention, including the steps of the methodologies described above, can be embedded in a computer program product. The computer program product can comprise a computer-readable storage medium in which is embedded a computer program comprising computer-executable code for directing a computing device or computer-based system to perform the various procedures, processes and methods described herein. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

The illustrations of embodiments described herein are intended to provide a general understanding of the structure of various embodiments, and they are not intended to serve as a complete description of all the elements and features of apparatus and systems that might make use of the structures described herein. Many other embodiments will be apparent to those of skill in the art upon reviewing the above description. Other embodiments may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

Thus, although specific embodiments have been illustrated and described herein, it should be appreciated that any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description. Therefore, it is intended that the disclosure not be limited to the particular embodiment(s) disclosed as the best mode contemplated for carrying out this invention, but that the invention will include all embodiments falling within the scope of the appended claims.

The Abstract of the Disclosure is provided to comply with U.S. Rule 37 C.F.R. §1.72(b), requiring an abstract that will allow the reader to quickly ascertain the nature of the technical disclosure. It is submitted with the understanding that it will not be used to interpret or limit the scope or meaning of the claims.

What is claimed is:

1. A method of tracking in a medical procedure, the method comprising:
providing a medical device (198) with at least one Optical Atomic Magnetometer (OAM) sensor (199) integrally connected thereto;
positioning the medical device into a target anatomy (105) of a patient;
applying a magnetic field (145) to the target anatomy;
detecting the magnetic field using the OAM sensor; and
determining a position of the medical device based on one or more parameters associated with the detected magnetic field.

2. The method of claim 1, further comprising generating the magnetic field (145) using a field generator (140) that is positioned external to the patient, wherein the magnetic field is spatially-encoded.

3. The method of claim 1, further comprising:
performing imaging of the target anatomy;
registering an OAM space of the target anatomy with an imaging space of the target anatomy; and
superimposing the determined position of the medical device (198) on the imaging of the target anatomy.

4. The method of claim 3, further comprising displaying the superimposed images in real-time.

5. The method of claim 3, further comprising performing the imaging using at least one of computed tomography, magnetic resonance imaging, and ultrasound imaging.

6. The method of claim 1, wherein the one or more parameters of the magnetic field (145) are an intensity and an orientation of the magnetic field.

7. The method of claim 1, further comprising providing signals representative of the one or more parameters to a processor (120) by way of transmission lines extending from the medical device (198), the processor determining the position of the medical device.

8. The method of claim 1, further comprising filtering out interference fields using one or more other OAM sensors positioned at a pre-determined distance from the magnetic field.

9. The method of claim 1, further comprising providing an imageable region (405) on the medical device.

10. A computer-readable storage medium in which computer-executable code is stored, the computer-executable code configured to cause a computing device, in which the computer-readable storage medium is provided, to:
determine a position of a medical device (198) in a target anatomy (105) of a patient based on one or more parameters associated with a magnetic field (145) applied to the target anatomy and detected using Optical Atomic Magnetometer (OAM) sensors (199).

11. The computer-readable storage medium of claim 10, further comprising computer-executable code for causing the computing device to:
detect the magnetic field (145) using the OAM sensors (210) positioned in proximity to the target anatomy (105) and external to the patient.

12. The computer-readable storage medium of claim 10, further comprising computer-executable code for causing the computing device to perform metal distortion compensation on the determined position of the medical device (198).

13. The computer-readable storage medium of claim 11, further comprising computer-executable code for causing the computing device to:
register an OAM space of the target anatomy (105) with an imaging space of the target anatomy; and
superimpose the determined position of the medical device (198) on imaging of the target anatomy.

14. The computer-readable storage medium of claim 13, further comprising computer-executable code for causing the computing device to display the superimposed images in real-time.

15. The computer-readable storage medium of claim 13, wherein the imaging is at least one of computed tomography, magnetic resonance imaging, and ultrasound imaging.

16. A tracking system for a target anatomy of a patient, the system comprising:
a medical device (298) having a magnetic source (299) integrally connected thereto and positionable in the target anatomy (105);
a plurality of Optical Atomic Magnetometer (OAM) sensors (210) for detecting a magnetic field (145) from the magnetic source; and
a processor (120) for determining a position of the medical device based on one or more parameters associated with the detected magnetic field.

17. The system of claim 16, wherein the magnetic source (299) is a permanent magnet material.

18. The system of claim 16, wherein the magnetic source (299) is positioned at an end of the medical device (298), and wherein the OAM sensors (210) are positionable on the patient.

19. The system of claim 16, further comprising an imaging modality (150) for capturing images of the target anatomy (105), wherein the processor (120) registers an imaging space with an OAM space.

20. The system of claim 19, further comprising a display device (130), wherein the images are displayed on the display device in real-time.

21. The system of claim 16, wherein the OAM sensor has a temperature control device for thermally insulating the patient from the OAM sensor.

22. A method of mapping, the method comprising:
introducing magnetic particles into a patient;
providing a medical device (198) with at least one Optical Atomic Magnetometer (OAM) sensor (199) integrally connected thereto;
positioning the medical device in proximity to a target anatomy (105) of the patient;
detecting a magnetic field (145) of the magnetic particles using the OAM sensor; and
mapping the target anatomy based on one or more parameters associated with the detected magnetic field.
